(19) United States
(12) Patent Application Publication
(10) Pub. No.: US 2013/0085510 A1
(43) Pub. Date: Apr. 4, 2013

(54) ROBOT-MOUNTED SURGICAL TABLES

(71) Applicants: David Stefanchik, Morrow, OH (US); Omar J. Vakharia, Cincinnati, OH (US)

(72) Inventors: David Stefanchik, Morrow, OH (US); Omar J. Vakharia, Cincinnati, OH (US)

(73) Assignee: ETHICON ENDO-SURGERY, INC., Cincinnati, OH (US)

(21) Appl. No.: 13/250,018

(22) Filed: Sep. 30, 2011

(51) Int. Cl. A61B 19/00; B25J 13/06; B25J 19/04; B25J 13/08; B25J 19/02

(52) U.S. Cl. 606/130; 700/245; 700/258; 700/259; 700/264; 901/30; 901/2

(57) ABSTRACT

The systems and methods disclosed herein generally involve a robotically-assisted surgical system in which a platform for supporting a patient is physically and operatively coupled to a surgical robot and an associated controller. As a result, the position of the patient can be controlled remotely using the robot, and the controller can have an awareness of the position and orientation of the patient with respect to the operating room and with respect to various components of the robot. Such systems can thus maintain a fixed frame of reference between the patient and one or more end effectors of the surgical robot, eliminating the need for recalibration of the system due to patient movement.

### FIELD

The present invention relates to robot-mounted surgical tables and methods of using the same.

### BACKGROUND

Minimally-invasive surgery (MIS) is often preferred over traditional open surgical procedures due to the reduced post-operative recovery time and minimal scarring associated therewith. Laparoscopic surgery is one type of MIS procedure in which one or more small incisions are formed in the abdomen of a patient and one or more trocars are inserted through the incisions to form pathways that provide access to the abdominal cavity. Endoscopic surgery is another type of MIS procedure in which elongate flexible shafts are introduced into the body through a natural orifice.

Various robotic systems have been developed to assist in MIS procedures. FIG. 1 illustrates a prior art robotically-assisted MIS system 10. As shown, the system 10 generally includes a control station 12 and a surgical robot 14. The control station 12 includes a controller and one or more master components 16, and is electronically coupled to the surgical robot 14 via one or more communication or signal lines 18, or via a wireless interface. The control station 12 can be positioned remotely from the surgical robot 14. The surgical robot 14 includes a plurality of surgical arms 20, each having a slave component or end effector 22 operatively coupled thereto. The robot 14 is mounted on a fixed support frame 24 which is attached to the floor 26 of an operating room.

In use, the surgical robot 14 is positioned in proximity to an operating table (not shown) on which a patient is positioned. The table can include buttons or other controls mounted thereto for adjusting a height and incline of the table. An operator seated at the control station 12 provides inputs to the controller by manipulating the master components 16 or interacting with a graphical user interface. The controller interprets these inputs and controls movement of the surgical robot 14 in response thereto. Thus, manipulation of the master components 16 by a user is translated into corresponding manipulations of the slave components 22, which perform a surgical procedure on the patient.

In a typical procedure, the position and/or orientation of the operating table can change frequently. These changes can be inadvertent (e.g., when the table is bumped by a member of the operating room staff or by the robot 14) or intentional (e.g., when it is necessary or desirable to reposition the patient to improve access to portions of the patient).

The operating table and the surgical robot 14 are independently-operable components, and there is no fixed frame of reference between the two, nor is there any communication or feedback loop between the two. As a result, the system 10 has no awareness of changes in the operating table's position or orientation, and must be manually calibrated to the actual table positioning at the beginning of a procedure and each time the table is moved. This calibration must be performed by operating room staff, is a time-consuming and cumbersome process, and usually requires all of the end effectors 22 to be removed from the patient and reinserted, which can increase the risk of patient infection or other surgical complications.

Furthermore, there is no way to control the position and orientation of the table (and thus the position and orientation of the patient) using the system 10. Rather, any changes in patient positioning must be carried out manually by operating room staff. This is a significant disadvantage, particularly when it is desirable to shift or otherwise move a patient in the middle of a surgery to obtain better access to a surgical site within the patient.

Accordingly, a need exists for improved robotically-assisted surgical systems.

### SUMMARY

The systems and methods disclosed herein generally involve a robotically-assisted surgical system in which a platform for supporting a patient is physically and operatively coupled to a surgical robot and an associated controller. As a result, the position of the patient can be controlled remotely using the robot, and the controller can have an awareness of the position and orientation of the patient with respect to the operating room and with respect to various components of the robot. Such systems can thus maintain a fixed frame of reference between the patient and one or more end effectors of the surgical robot, eliminating the need for recalibration of the system due to patient movement.

In one aspect, a robotic apparatus is provided that includes at least one remotely-controlled arm having an end effector coupled thereto, a remotely-controlled patient support table for supporting a patient, and a controller configured to adjust a position and orientation of the end effector in response to changes in a position and orientation of the patient support table, such that a fixed frame of reference is maintained between the end effector and the patient support table.

The controller can be configured to adjust the position and orientation of the patient support table. The at least one remotely-controlled arm can include a plurality of remotely-controlled arms, each of the plurality of remotely controlled arms having an end effector coupled thereto and having a position and orientation that is adjustable by the controller. The apparatus can also include at least one input device configured to receive user input indicative of desired movement of at least one of the end effector and the patient support table and configured to communicate the received user input to the controller. The at least one input device can be positioned remotely from the at least one remotely-controlled arm and the patient support table. The patient support table can include a plurality of sections configured to move relative to one another.

In some embodiments, the at least one remotely-controlled arm and the patient support table can be coupled to a support frame. The patient support table can be movable with at least six degrees of freedom relative to the support frame. The support frame can be configured to be mounted to a ceiling.

The apparatus can also include a sensor system configured to measure a position and orientation of the patient support table relative to the support frame. The sensor system can include a plurality of sensors positioned on at least one of the patient support table and the support frame. The apparatus can also include an output device configured to display at least one of an image a surgical site, an image of the support frame and the patient support table, and a rendering of the support frame and the patient support table.

In another aspect, a surgical system is provided that includes a surgical robot having a slave assembly and a patient-receiving platform. The system also includes a first input device positioned remotely from the surgical robot, the first input device being configured to provide platform movement information to a controller in response to input received from a user. A position and orientation of the platform can be robotically-adjustable in response to one or more platform control signals generated by the controller, the one or more platform control signals being generated based on the platform movement information.

The system can also include a second input device positioned remotely from the surgical robot, the second input device being configured to provide slave assembly movement information to the controller in response to input received from a user. A position and orientation of the slave assembly can be robotically-adjustable in response to one or more slave assembly control signals generated by the controller, the one or more slave assembly control signals being generated based on the slave assembly movement information.

In one embodiment, the controller can be configured to automatically generate slave assembly control signals when platform control signals are generated, the slave assembly control signals being effective to cause movement of the slave assembly that corresponds to movement of the platform caused by the platform control signals.

In another aspect, a method of performing robotically-assisted surgery using a robot having a surgical arm and a patient-receiving platform is provided. The method includes receiving user input indicative of desired movement of the platform, generating control signals based on the user input that instruct the robot to effect a change in position or orientation of the platform, and generating control signals based on the user input that instruct the robot to effect a corresponding change in position or orientation of the surgical arm, such that a fixed frame of reference is maintained between the platform and the surgical arm when the platform is moved.

The user input can be received by an input device positioned remotely from the robot. The method can also include calculating a position and orientation of the platform relative to the surgical arm based on the output of one or more sensors.

### BRIEF DESCRIPTION OF THE DRAWINGS

The presently disclosed systems and methods will be more fully understood from the following detailed description taken in conjunction with the accompanying drawings, in which:

FIG. 1 is a perspective view of a prior art robotically-assisted surgical system;

FIG. 2 is a diagram of the six degrees of freedom of a rigid body;

FIG. 3 is a perspective view of one embodiment of a robotically-assisted surgical system that includes an integrated surgical platform;

FIG. 4 is a schematic diagram of the system of FIG. 3; and

FIG. 5 is a perspective view of another embodiment of a robotically-assisted surgical system that includes an integrated surgical platform.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

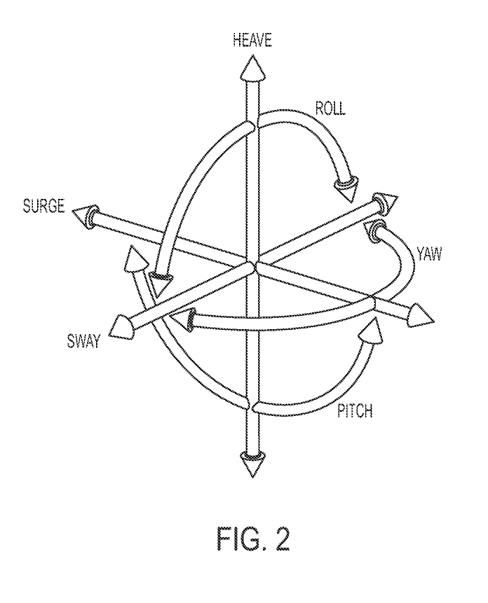

There are a number of ways in which to describe an object's position and orientation in space. For example, the position and orientation of an object can be characterized in terms of the object's degrees of freedom. The degrees of freedom of an object are the set of independent variables that completely identify the object's position and orientation. As shown in FIG. 2, the six degrees of freedom of a rigid body with respect to a particular Cartesian reference frame can be represented by three translational (position) variables (e.g., surge, heave, and sway) and by three rotational (orientation) variables (e.g., roll, pitch, and yaw).

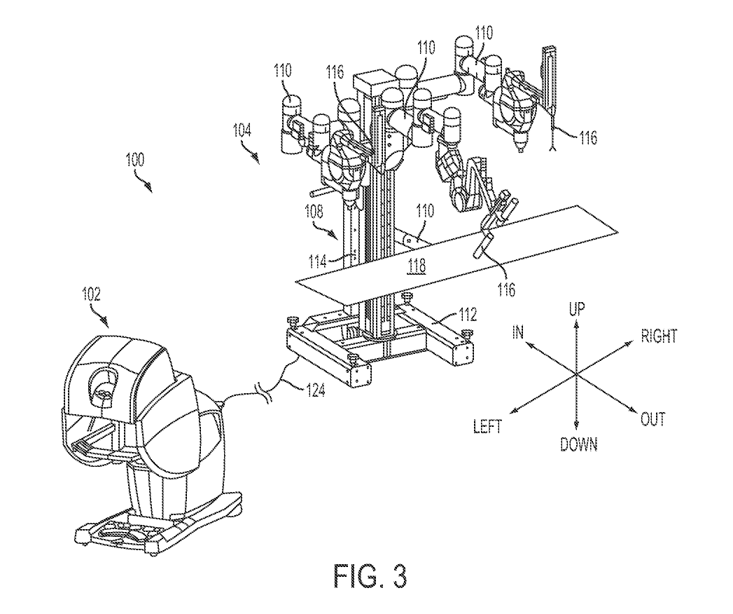

For convenience of description, surge is sometimes described herein as translational movement in an “in” direction or an “out” direction, heave is sometimes described as translational movement in an “up” direction or a “down” direction, and sway is sometimes described as translational movement in a “left” direction or a “right” direction Likewise, roll is sometimes described herein as rotation about an in-out axis, pitch is sometimes described as pivoting in the up direction or the down direction, and yaw is sometimes described as pivoting in the left direction or the right direction. An exemplary mapping of the in, out, up, down, left, and right directions to a surgical system is shown in FIG. 3. This mapping is generally used throughout the description that follows, for example to describe the relative positioning of components of the system (e.g., “upper,” “lower,” “left,” “right”) or to describe direction of movement within a particular degree of freedom (e.g., “leftwards,” “rightwards,” “up,” “down”). This terminology and the illustrated mapping are not intended to limit the invention, and a person having ordinary skill in the art will appreciate that these directional terms can be mapped to the system or any component thereof in any of a variety of ways.

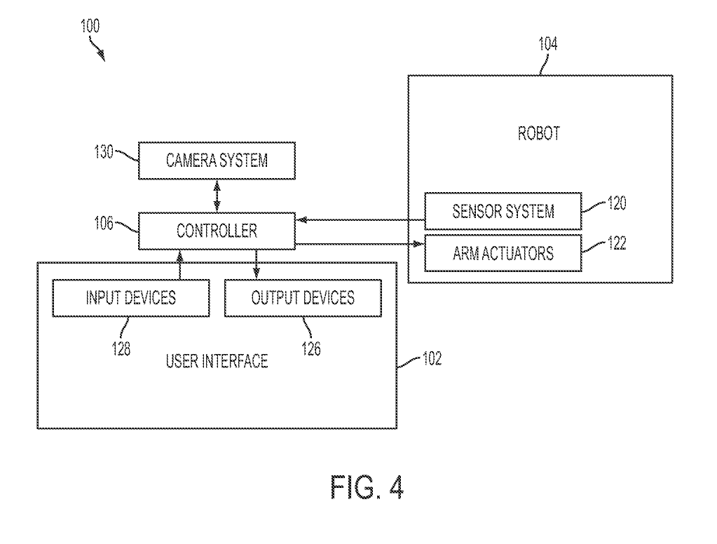

FIGS. 3 and 4 illustrate one exemplary embodiment of robotically-assisted surgical system 100. The system generally includes a user interface 102 and a surgical robot 104 (also referred to herein as a robotic apparatus). The system 100 also includes a controller 106 which can be a component of the user interface 102, a component of the surgical robot 104, and/or a plurality of components distributed across the user interface 102, the robot 104, and/or any of a variety of other systems.

The surgical robot 104 can include a support frame 108 having a plurality of surgical arms 110 coupled thereto. The support frame 108 can be fixedly positioned within the operating room, for example by being mounted directly to the floor, ceiling, or one or more walls of the operating room. In the illustrated embodiment, the support frame 108 includes a base 112 and an upright member 114 that extends vertically therefrom.

The surgical arms can 110 include a plurality of sections, which can be coupled to one another and/or to the support frame 108 by any of a variety of joints (e.g., pivot joints, rotation joints, universal joints, wrist joints, continuously variable joints, and so forth). The surgical arms 110 can also include one or more linkages or actuators 122 (e.g., gears, cables, servos, magnets, counterweights, motors, hydraulics, pumps, and the like) which can be manipulated by the controller 106 to effect movement of the arms 110 and/or actuation of end effectors 116 coupled thereto. For example, in the case of a grasper-type end effector having two opposed jaws, one or more servo-driven cables can be provided such that the jaws can be opened and closed by the controller 106. The surgical arms 110 can thus be controlled to allow the position and orientation in space of an end effector 116 or other object coupled thereto to be adjusted. Such adjustments can be made manually by operating room staff or by a remote user via the user interface 102 and the controller 106, as described below.

Any of a variety of end effectors 116 can be mated to the surgical arms 110. Exemplary end effectors include graspers, dissectors, needle drivers, cameras, light sources, and the like. In embodiments in which a camera end effector is provided, the camera can be configured to capture images of a surgical site, such as the interior of a body cavity of a patient. The captured images can be transmitted in real time (e.g., as a live video feed) to the user interface 102 for viewing by a user.

In addition, a patient-receiving surgical platform 118 can be coupled to one or more of the surgical arms 110. The surgical platform 118 can be substantially rectangular and can be configured to support a patient on which a surgical procedure is to be performed using the robot 104. The surgical platform 118 can optionally be formed from a plurality of sections, which can each be independently adjustable relative to the other sections to provide additional control over patient positioning (e.g., to move a particular area of the patient's body, such as the head or legs). The arm 110 to which the platform 118 is coupled can be configured to maintain the platform in a fixed position and orientation relative to the support frame 108, or can allow the platform 118 to move with one or more degrees of freedom relative thereto (e.g., with at least six degrees of freedom). Thus, in one embodiment, translational movement (heave, surge, and sway) and rotational movement (roll, yaw, and pitch) of the platform 118 relative to the support frame 108 (and thus relative to the operating room) can be achieved.

The robot 104 can also include a sensor system 120 configured to provide closed loop feedback as to the position and orientation of the surgical platform 118 and/or of the various end effectors 116. This can allow the controller 106 to confirm its understanding of the position and orientation of such components by determining the actual position and orientation of the components using one or more sensors. In one embodiment, the sensor system 120 can include a plurality of cameras configured to capture images of the robot 104 and an image processing module configured to determine the relative positioning of the various components based on the captured images. In another embodiment, the sensor system 120 can include a plurality of sensors positioned at various points on the surgical robot 104 and configured to generate output signals indicative of robot positioning. For example, sensors configured to detect motion, position, or angles can be mounted to the surgical arms 110 or the joints thereof to provide sensor data which can be processed by the controller 106 to calculate a position and orientation of the platform 118 and/or end effectors 116. The sensor data can also be used to create a 3D rendering of the respective positions and orientations of the surgical arms 110, end effectors 116, platform 118, etc., which can be displayed to a user via the user interface 102.

Because movement of the platform 118 can be controlled by the controller 106 without intervention by human operating room staff, the controller can have an awareness of the position and orientation of the platform 118 relative to the support frame 108 or other components of the robot 104 (e.g., the surgical arms 110 to which the various end effectors 116 are coupled). The controller 106 can also obtain this awareness using the sensor system 120 described above. The controller 106 can thus be configured to maintain a fixed frame of reference between the platform 118 and one or more of the end effectors 116. For example, in one embodiment, when the position or orientation of the platform 118 is adjusted, the controller 106 can automatically make corresponding adjustments to the position or orientation of one or more of the end effectors 116.

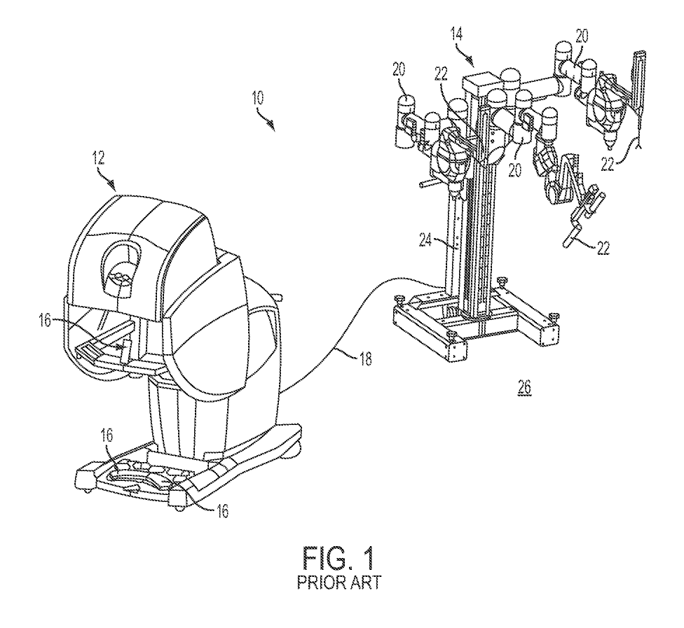

It will thus be appreciated that movement of the platform 118, whether inadvertent or intentional, can be detected by the controller 106 and compensated for, without the need for the cumbersome and complicated recalibration procedures required in systems of the type illustrated in FIG. 1. It will further be appreciated that movement of the platform 118 (and thus movement of the patient) can be controlled remotely, without assistance from human operating room personnel, in a manner similar to that used to remotely control movement of the end effectors 116.

The controller 106 can include one or more computer systems (e.g., personal computers, workstations, server computers, desktop computers, laptop computers, tablet computers, or mobile devices), and can include functionality implemented in software, hardware, or combinations thereof. A computer system can include one or more processors which can control the operation of the computer system. A computer system can also include one or more memories, which can provide temporary storage for code to be executed by the processors or for data acquired from one or more users, storage devices, and/or databases. Computer systems can also include network interfaces and storage devices. Network interfaces can allow the computer system to communicate with remote devices (e.g., other computer systems) over a network. Storage devices can include any conventional medium for storing data in a non-volatile and/or non-transient manner, such as hard disk drives, flash drives, USB drives, optical drives, various media cards, and/or any combination thereof. It will be appreciated that the elements of a computer system described herein are merely exemplary, that they can be some or all of the elements of a single physical machine, and that not all of the elements need to be located on or in the same physical machine or enclosure.

The user interface 102 and the surgical robot 104 can be operatively coupled to the controller 106, either wirelessly or via one or more electrical communication or transmission lines 124, such that a user can operate the surgical robot 104 from a remote location. The remote location can be an opposite side of the operating room, a room that is separated from the operating room, or any other location in which electronic communication can be established between the user interface 102 and the controller 106 or surgical robot 104 (e.g., using the Internet or some other computer network).

The user interface 102 can include one or more output devices 126 (e.g., one or more display screens) and one or more input devices 128 (e.g., keyboards, pointing devices, joysticks, or surgical handles).

The input devices 128 can allow a user to control the behavior of the surgical robot 104, such as movement of the surgical arms 110 (and the platform 118 and end effectors 116 coupled thereto). The output devices 126 can provide feedback to the user, such as image or video of the operating room and/or a surgical site.

The input devices 128 can include a platform adjustment device for adjusting the position and orientation of the surgical platform 118. A user's manipulation of the platform adjustment device can be interpreted by one or more sensors coupled to the controller 106, and can be translated by the controller into control signals which can then be communicated to the surgical robot 104 to effect corresponding movement of the surgical platform 118. This functionality can eliminate the need for medical personnel in the operating room to manually adjust the surgical platform 118 and gives a user direct control over patient positioning. Preferably, at least one output device 126 is configured to display a real-time video feed of the surgical platform 118 while the platform adjustment device is being manipulated so that a user can see changes in the platform's position and orientation. Thus, the system 100 can include a camera system 130 including one or more cameras positioned in the operating room and focused on the support frame 108, the surgical arms 110, and/or the surgical platform 118. The field of view and focus of the camera system 130 can also be adjusted by the controller 106. When the surgical platform 118 is moved, the controller 106 can optionally be configured to command the robot 104 to move the surgical arms 110 to which end effectors 116 are coupled in a corresponding fashion. This can advantageously maintain a fixed positional and/or orientational relationship between said surgical arms 110 and the platform 118 before, during, and after platform movement. In other words, a fixed frame of reference can be maintained between the patient and one or more of the end effectors 116.

The input devices 128 can also include one or more end effector adjustment devices configured to move or otherwise manipulate any of the end effectors 116 of the surgical robot 104. Thus, in one embodiment, a user can engage one or more surgical handle input devices 128 of the user interface 102 while observing the surgical site on an output device 126 of the user interface 102. The user's manipulation of the surgical handle input devices 128 can be interpreted by one or more sensors coupled to the controller 106, and can be translated by the controller into control signals which can then be communicated to the surgical robot 104 to effect corresponding manipulations of one or more of the end effectors 116.

In one embodiment, the controller 106 can be configured to automatically move the platform 118 in response to user instructions to move one or more of the end effectors 116. For example, a user may be focused on a specific surgical site shown on an output device 126 of the user interface 102 and may be unaware of the extracorporeal positioning of the surgical arms 110. The user may thus attempt to move an end effector 116 in such a way that the arm 110 to which the end effector is mounted would need to move beyond its capable range of motion. In this case, the controller 106 can be configured to automatically reposition the platform 118 to allow the desired motion to be achieved.

An exemplary method for performing this type of compensation is as follows. First, the controller 106 can receive a user input and calculate the arm movement necessary to obtain the desired end effector positioning. The controller 106 can then determine whether the necessary arm movement would exceed the range of motion of any of the surgical arms 110. If such movement would not exceed the range of motion of any of the arms 110, the robot 104 can be commanded to carry out the desired movement. If the movement would exceed the range of motion of one or more of the arms 110, the controller 106 can command the robot 104 to move the platform 118 relative to one or more of the end effectors 116 such that the desired movement can be achieved. The controller 106 can then recalculate the necessary arm movement based on the new platform position, and instruct the robot 104 to carry out the required movement. This compensation technique can allow desired movements that would otherwise be impossible (e.g., due to a limitation in the robot's range of motion) to be achieved without having to manually disengage the robot 104 from the patient, reposition the patient, and recalibrate the entire system 100.

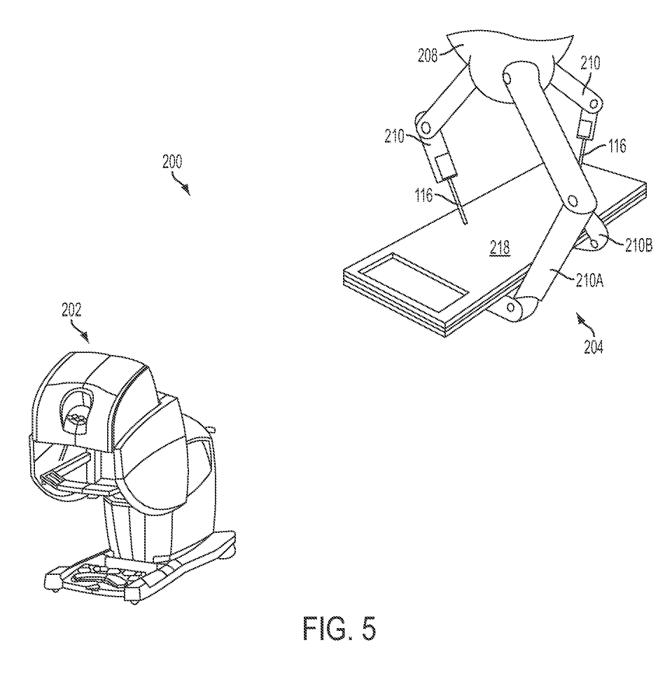

FIG. 5 illustrates another embodiment of a robotically-assisted surgical system 200 that includes a user interface 202, a surgical robot 204, and a controller (not shown). In the system 200, the support frame 208 is mounted to the ceiling of an operating room and the surgical platform 218 is mounted to first and second robotic arms 210A, 210B. The ceiling-mounted nature of this embodiment can advantageously provide more usable space around the platform 218 for medical personnel and equipment, and can reduce the amount of sterile draping required. Any of the features described above with respect to the system of FIGS. 3-4 can be applied to the system of FIG. 5. For example, as shown, the system 200 can include additional surgical arms 210 having end effectors 216 coupled thereto.

In use, the systems described herein can allow a surgeon or other user to perform a robotically-assisted surgical procedure. In one embodiment, a patient can be placed on the surgical platform 118 and coupled thereto (e.g., using straps, collars, and so forth) such that the patent's position and orientation is substantially fixed relative to the surgical platform 118. One or more incisions can then be formed in the patient and trocars can be inserted therein to provide one or more access channels to a surgical site within the patient. The end effectors 116 of the surgical arms 110 can then be passed through the trocars and placed in proximity to the surgical site, either manually or under control of the robot 104.

A user positioned remotely from the robot 104 can then operate the user interface 102 to provide inputs to the controller 106 (e.g., by manipulating the input devices 128). These inputs can be interpreted by the controller 106 and translated into control instructions for the surgical robot 104, which can carry out end effector 116 movements or actuation based on the control instructions in order to carry out the surgical procedure. The user can also view the surgical site and/or operating room on an output device 126 of the user interface 102.

During a procedure, the position and/or orientation of the platform 118 (and the patient coupled thereto) can be robotically adjusted using the platform adjustment input device. For example, a surgeon operating in the pelvic cavity may want to adjust the pitch of the platform 118 such that the patient's torso and head are tilted downwards, allowing gravity to shift the patient's internal organs away from the pelvic cavity. To do so, the surgeon can actuate a platform adjustment input device (e.g., a joystick) to instruct the robot 104 to change the pitch of the platform 118. Accordingly, patient position and orientation can be adjusted using the robot 104, without manual intervention by operating room staff. When changes in patient position or orientation occur, the controller 106 can automatically recalibrate the robot 104 to the changed position or orientation. Thus, in the example above, the robot 104 can automatically adjust the pitch of the other surgical arms 110 and/or end effectors 116 in correspondence with the pitch adjustment made to the platform 118. In other words, because the platform 118 and the other components of the robot 104 are physically and operatively coupled to one another, the controller 106 has an awareness of their relative position and orientation, which allows the controller 106 to compensate for movement of one by moving the other in a corresponding fashion. This allows the patient position and orientation to be adjusted without requiring removal of the end effectors 116 from the trocars, manual recalibration of the robot 104, and subsequent reinsertion of the end effectors into the trocars.

If, during the course of a procedure, a user attempts a movement that would otherwise require one or more of the surgical arms 110 to move beyond their range of motion, the robot 104 can automatically reposition the platform 118 such that the movement can be achieved without exceeding the range of motion of the arms 110, as explained above.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A robotic apparatus, comprising;
at least one remotely-controlled arm having an end effector coupled thereto;
a remotely-controlled patient support table for supporting a patient; and
a controller configured to adjust a position and orientation of the end effector in response to changes in a position and orientation of the patient support table, such that a fixed frame of reference is maintained between the end effector and the patient support table.

2. The system of claim 1, wherein the controller is configured to adjust the position and orientation of the patient support table.

3. The system of claim 1, wherein the at least one remotely-controlled arm comprises a plurality of remotely-controlled arms, each of the plurality of remotely controlled arms having an end effector coupled thereto and having a position and orientation that is adjustable by the controller.

4. The system of claim 1, further comprising at least one input device configured to receive user input indicative of desired movement of at least one of the end effector and the patient support table and configured to communicate the received user input to the controller.

5. The system of claim 4, wherein the at least one input device is positioned remotely from the at least one remotely-controlled arm and the patient support table.

6. The system of claim 1, wherein the patient support table includes a plurality of sections configured to move relative to one another.

7. The system of claim 1, wherein the at least one remotely-controlled arm and the patient support table are coupled to a support frame.

8. The system of claim 7, wherein the patient support table is movable with at least six degrees of freedom relative to the support frame.

9. The system of claim 7, wherein the support frame is configured to be mounted to a ceiling.

10. The system of claim 7, further comprising a sensor system configured to measure a position and orientation of the patient support table relative to the support frame.

11. The system of claim 10, wherein the sensor system includes a plurality of sensors positioned on at least one of the patient support table and the support frame.

12. The system of claim 7, further comprising an output device configured to display at least one of an image a surgical site, an image of the support frame and the patient support table, and a rendering of the support frame and the patient support table.

13. A surgical system comprising:
a surgical robot having a slave assembly and a patient-receiving platform; and
a first input device positioned remotely from the surgical robot, the first input device being configured to provide platform movement information to a controller in response to input received from a user;
wherein a position and orientation of the platform is robotically-adjustable in response to one or more platform control signals generated by the controller, the one or more platform control signals being generated based on the platform movement information.

14. The system of claim 13, further comprising:
a second input device positioned remotely from the surgical robot, the second input device being configured to provide slave assembly movement information to the controller in response to input received from a user;
wherein a position and orientation of the slave assembly is robotically-adjustable in response to one or more slave assembly control signals generated by the controller, the one or more slave assembly control signals being generated based on the slave assembly movement information.

15. The system of claim 14, wherein the controller is configured to automatically generate slave assembly control signals when platform control signals are generated, the slave assembly control signals being effective to cause movement of the slave assembly that corresponds to movement of the platform caused by the platform control signals.

16. A method of performing robotically-assisted surgery using a robot having a surgical arm and a patient-receiving platform, the method comprising:
receiving user input indicative of desired movement of the platform;
generating control signals based on the user input that instruct the robot to effect a change in position or orientation of the platform; and
generating control signals based on the user input that instruct the robot to effect a corresponding change in position or orientation of the surgical arm, such that a fixed frame of reference is maintained between the platform and the surgical arm when the platform is moved.

17. The method of claim 16, wherein the user input is received by an input device positioned remotely from the robot.

18. The method of claim 16, further comprising calculating a position and orientation of the platform relative to the surgical arm based on the output of one or more sensors.
